(19) United States
(12) Patent Application Publication
(10) Pub. No.: US 2024/0189245 A1
(43) Pub. Date: Jun. 13, 2024

(54) CYCLOSPORINE COMPOSITIONS AND METHODS OF USE THEREOF

(71) Applicant: ProTransit Nanotherapy LLC, Omaha, NE (US)

(72) Inventors: Gary L. Madsen, Omaha, NE (US); Christopher L. Porter, Omaha, NE (US)

(21) Appl. No.: 18/287,762

(22) PCT Filed: Jun. 14, 2022
(86) PCT No.: PCT/US22/33404

§ 371 Date: Oct. 20, 2023

Related U.S. Application Data
(60) Provisional application No. 63/210,250, filed on Jun. 14, 2021.

(51) Int. Cl. A61K 9/51; A61K 38/13; A61K 45/06; A61P 17/06

(52) U.S. Cl. CPC A61K 9/5153; A61K 9/5123; A61K 9/5138; A61K 38/13; A61K 45/06; A61P 17/06

(57) ABSTRACT

Cyclosporine containing nanoparticles and methods for making and using the same are provided.

This application is claims priority under 35 U.S.C. § 119(e) to U.S. Provisional Patent Application No. 63/210,250, filed on Jun. 14, 2021. The foregoing application is incorporated by reference herein.

### FIELD OF THE INVENTION

The present invention relates to cyclosporine particles and methods of use thereof.

### BACKGROUND OF THE INVENTION

Cyclosporine is a very effective immunosuppressant medication and is known to be able to treat a broad spectrum of diseases such as arthritis (Salvarani et al. (2001) J. Rheumatol., 28(10):2274-82), alopecia (Jang et al. (2016) Ann. Dermatol., 28(5): 569-574), psoriasis (Rosmarin et al. (2010) J. Am. Acad. Dermatol., 62(5):838-53), and dry eye (Perry et al. (2008) Arch. Ophthalmol., 126(8): 1046-50). This wide range of therapeutic uses requires efficient delivery systems with sustained release of the drug. Currently available formulations of cyclosporine are only able to serve this purpose to a limited extent due to the constraints of the physical properties of cyclosporine such as high molecular weight, low solubility, low permeability, and narrow therapeutic index of cyclosporine.

Therefore, it is clear that improved delivery systems for cyclosporine are needed.

### SUMMARY OF THE INVENTION

In accordance with the instant invention, methods of treating, inhibiting, and/or preventing diseases and disorders treatable with cyclosporine are provided. In a particular embodiment, the method comprises topically administering at least one nanoparticle (e.g., to the skin or eye) to a subject in need thereof, wherein the nanoparticle comprises at least one biodegradable polymer and cyclosporine. The biodegradable polymer may be, for example, poly (lactide-co-glycolide) (e.g., poly(DL-lactide-co-glycolide)), polylactide, or derivatives thereof. The nanoparticle may further comprise at least one plasticizer (e.g., dimethyl tartrate). The methods of the instant invention may also comprise the administration of at least one other therapeutic agent. The nanoparticles may be administered using a suitable carrier for topical application (e.g., lotion, cream, haircare product, etc.).

In accordance with another aspect of the instant invention, compositions (e.g., topical compositions) are provided which are well-suited for the delivery of cyclosporine. In a particular embodiment, the composition comprises at least one carrier (e.g., a carrier acceptable for topical delivery (e.g., a pharmaceutically and/or cosmetically acceptable carrier)) and nanoparticles comprising cyclosporine.

### BRIEF DESCRIPTIONS OF THE DRAWINGS

FIG. 1 provides a release profile of cyclosporine from poly-lactic-co-glycolic acid (PLGA) particles.

FIG. 2 provides images of an imiquimod induced plaque psoriasis mouse model wherein the mice were subsequently topically treated with vehicle (control; top) or cyclosporine A PLGA particles (Pro-NP™) at with low dose (0.125%; middle) or high dose (0.25%; bottom).

FIG. 3 provides a graph of the PASI (psoriasis are and severity), erythema (redness), induration (thickness), and desquamation (scaling) for a subject over time treated with cyclosporine A particles daily.

FIG. 4 provides images of hematoxylin and eosin (H & E) staining of skin biopsies from patient 3 prior to treatment (top) and after treatment (bottom) with cyclosporine A loaded particles. Psoriasiform hyperplasia with associated inflammation is seen prior to treatment with a loss of psoriasiform and more normal epidermis after treatment.

### DETAILED DESCRIPTION OF THE INVENTION

Improved drug delivery may be achieved by using nanoparticles. The small size and increased surface area of nanoparticles enables a close and extended contact with a surface, e.g., with the stratum corneum for topical application. Moreover, nanoparticles allow for controlled drug release which will lead to deeper penetration of the drug while minimizing both the required drug dosage and drug losses. The use of nanoparticles will also reduce adverse effects while increasing therapeutic efficacy.

In accordance with one aspect of the instant invention, nanoparticles which encapsulate cyclosporine (e.g., cyclosporine A or a pharmaceutically acceptable salt thereof) are provided. The nanoparticles of the instant invention stabilize the encapsulated compound, allow the penetration of cyclosporine (e.g., through the skin layers and into hair follicles (e.g., past the sebum plug)), and deliver cyclosporine over a sustained period of time. The nanoparticles of the instant invention can also be completely metabolized by the body in a non-toxic manner.

The nanoparticles of the instant invention provide a superior drug delivery system that is able to: (1) increase cyclosporine's stability, (2) improve the pharmacokinetic and/or pharmacodynamic profiles of cyclosporine, (3) increase the permeation and formation of skin depots in the stratum corneum, epidermis, and/or follicular bulb, (4) promote therapeutic adherence, and/or (5) decrease toxicity and/or treatment resistance to cyclosporine.

In accordance with the instant invention, methods of delivering cyclosporine are provided. The methods of the instant invention comprise administering (e.g., topically) at least one nanoparticle of the instant invention (or a composition comprising at least one nanoparticle) comprising or encapsulating cyclosporine to a subject in need thereof. The nanoparticles of the instant invention may be used to treat, inhibit, and/or prevent any disease or disorder for which cyclosporine is therapeutic. The nanoparticles of the instant invention may be administered by any means (e.g., topically, orally, intravenously, etc.). In a particular embodiment, the nanoparticles of the instant invention are administered topically such as to the eye or skin.

In accordance with another aspect of the instant invention, methods of treating, inhibiting, and/or preventing hair loss and/or related disorders are provided. Examples of hair loss disorders include, without limitation: alopecia, alopecia areata, androgenetic alopecia (alopecia androgenetica), hypotrichosis (e.g., of the eyelash or eyebrow) and hair miniaturization. The methods of the instant invention comprise administering (particularly topically) at least one nanoparticle of the instant invention (or a composition comprising at least one nanoparticle) comprising or encapsulating cyclosporine to a subject in need thereof. In a particular embodiment, the nanoparticle is administered to the skin. In a particular embodiment, the methods deliver the compound across the sebum plug. The methods may further comprise the administration of at least one other therapeutic agent for the treatment, inhibition, or prevention of hair loss and/or related disorders (e.g., hair regrowth agent and/or antioxidant). The additional therapeutic agent may be administered in a separate composition from the nanoparticles of the instant invention. The compositions may be administered at the same time or at different times (e.g., sequentially).

In accordance with another aspect of the instant invention, methods of treating, inhibiting, and/or preventing dry eye are provided. The methods of the instant invention comprise administering (particularly topically) at least one nanoparticle of the instant invention (or a composition comprising at least one nanoparticle) comprising or encapsulating cyclosporine to a subject in need thereof. In a particular embodiment, the nanoparticle is administered to the eye (e.g., ocularly). In a particular embodiment, the dry eye disease is mild, moderate, or severe. The methods may further comprise the administration of at least one other agent for the treatment of dry eye disease. The additional agent may be administered in a separate composition from the nanoparticles of the instant invention. The compositions may be administered at the same time or at different times (e.g., sequentially).

In accordance with another aspect of the instant invention, methods of treating, inhibiting, and/or preventing psoriasis (e.g., plaque psoriasis) are provided. The methods of the instant invention comprise administering (particularly topically) at least one nanoparticle of the instant invention (or a composition comprising at least one nanoparticle) comprising or encapsulating cyclosporine to a subject in need thereof. In a particular embodiment, the nanoparticle is administered to the skin. In a particular embodiment, the psoriasis is mild, moderate, or severe. The methods may further comprise the administration of at least one other agent for the treatment of psoriasis. The additional agent may be administered in a separate composition from the nanoparticles of the instant invention. The compositions may be administered at the same time or at different times (e.g., sequentially).

In accordance with another aspect of the instant invention, methods of treating, inhibiting, and/or preventing arthritis are provided. The methods of the instant invention comprise administering (particularly topically) at least one nanoparticle of the instant invention (or a composition comprising at least one nanoparticle) comprising or encapsulating cyclosporine to a subject in need thereof. In a particular embodiment, the nanoparticle is administered to the skin. In a particular embodiment, the arthritis is rheumatoid arthritis or psoriatic arthritis. The methods may further comprise the administration of at least one other agent for the treatment of arthritis. The additional agent may be administered in a separate composition from the nanoparticles of the instant invention. The compositions may be administered at the same time or at different times (e.g., sequentially).

The nanoparticles of the instant invention comprise at least one polymer and at least one encapsulated compound. Generally, the nanoparticle ranges in size from between 1 nm and 1000 nm, particularly between 1 nm and about 350 nm or between 1 nm and about 250 nm. While the instant invention generally describes the use of cyclosporine in the nanoparticles, it is also within the scope of the instant invention to use other therapeutic agents or compounds of interest in the nanoparticles (e.g., in combination with cyclosporin). Such agents or compounds include, without limitation, polypeptides, proteins, peptides, glycoproteins, nucleic acids (DNA, RNA, oligonucleotides, plasmids, siRNA, etc.), synthetic and natural drugs, polysaccharides, lipids, and the like.

In a particular embodiment, the polymer of the nanoparticles is a biocompatible and biodegradable polymer. The polymer may be a homopolymer or a copolymer. The polymer may be hydrophobic, hydrophilic, or amphiphilic. If the polymer is a copolymer, it may be a diblock, triblock, or multiblock copolymer. In a particular embodiment, the segments of the block copolymer comprise about 10 to about 500 repeating units, about 20 to about 300 repeating units, about 20 to about 250 repeating units, about 20 to about 200 repeating units, or about 20 to about 100 repeating units. Suitable polymers include, without limitation: poly(lactide-co-glycolides) (e.g., PLGA, PLLGA, etc.), poly(lactic acid), poly(alkylene glycol), polybutylcyanoacrylate, poly(methylmethacrylate-co-methacrylic acid), poly-allylamine, polyanhydride, polyhydroxybutyric acid, polyorthoesters, and the like. In particular embodiments, a nanoparticle is composed of a copolymer comprising at least one poly(lactic acid) segment and at least one poly(glycolic acid) segment. In a particular embodiment, the polymer is a poly (lactide-co-glycolide), particularly poly (D,L-lactide-co-glycolide) (PLGA). Examples of biocompatible polymers include, without limitation: natural or synthetic polymers such as polystyrene, polylactic acid, polyketal, butadiene styrene, styreneacrylic-vinyl terpolymer, polymethylmethacrylate, polyethylmethacrylate, polyalkylcyanoacrylate, styrene-maleic anhydride copolymer, polyvinyl acetate, polyvinylpyridine, polydivinylbenzene, polybutyleneterephthalate, acrylonitrile, vinylchloride-acrylates, polycaprolactone, poly(alkyl cyanoacrylates), poly(lactic-co-glycolic acid), and the like. Examples of natural polymers include polypeptides including those modified non-peptide components, such as saccharide chains and lipids; nucleotides; sugar-based biopolymers such as polysaccharides; cellulose; carbohydrates and starches; dextrans; lignins; polyamino acids; adhesion proteins; lipids and phospholipids (e.g., phosphorylcholine). In a particular embodiment, the polymer is poly(lactic-co-glycolic acid).

The nanoparticles of the present invention can further contain a polymer that affects the charge or lipophilicity or hydrophilicity of the particle. Any biocompatible polymer can be used for this purpose, including but not limited to, poly(vinyl alcohol).

The nanoparticles of the present invention can further comprise a plasticizer. The plasticizer may facilitate sustained release of the encapsulated compound by maintaining the structure of the nanoparticle. A plasticizer may be added to the nanoparticles to maintain the glass transition temperature above 37° C. despite a decline in molecular weight of the polymer with time. Without being bound by theory, the addition of the plasticizer allows for pores in the nanoparticle to remain open and facilitate a continuous release of the encapsulated compound. Suitable plasticizers are generally inert, non-toxic, and biocompatible. Plasticizers include, without limitation, triethyl citrate (e.g., Citroflex®, Morflex Inc., Greensboro, N.C.), glyceryl triacetate (e.g., triacetin), L-tartaric acid dimethyl ester (dimethyl tartrate, DMT), benzoates (e.g. terephthalates such as dioctyl terephthalate/DEHT, 1,2-cyclohexane dicarboxylic acid diisononyl ester (Hexamoll® DINCH®), epoxidized vegetable oils, alkyl sulphonic acid phenyl ester (ASE), sulfonamides (e.g. N-ethyl toluene sulfonamide (o/p ETSA), ortho and para isomers, N-(2-hydroxypropyl) benzene sulfonamide (HP BSA), N-(n-butyl) benzene sulfonamide (BBSA-NBBS)), organophosphates (e.g., tricresyl phosphate (TCP), tributyl phosphate (TBP)), glycols/polyethers, triethylene glycol (e.g., dihexanoate (3G6, 3GH), tetraethylene glycol diheptanoate (4G7)), polymeric plasticizer (e.g. polybutene), and bio-based plasticizers. Bio-based plasticizers may have better biodegradability and fewer biochemical effects and include, without limitation: acetylated monoglycerides, alkyl citrates, triethyl citrate (TEC), acetyl triethyl citrate (ATEC), tributyl citrate (TBC), acetyl tributyl citrate (ATBC), trioctyl citrate (TOC), acetyl trioctyl citrate (ATOC), trihexyl citrate (THC), acetyl trihexyl citrate (ATHC), butyryl trihexyl citrate (BTHC, trihexyl o-butyryl citrate), andrimethyl citrate (TMC). In a particular embodiment, the nanoparticles comprise the plasticizer triethyl citrate. In a particular embodiment, the nanoparticles comprise the plasticizer dimethyl tartrate (DMT) or tartaric acid. The amount of plasticizer employed in a nanoparticle can range from about 5 to about 40 weight percent of the nanoparticle, particularly from about 10 to 20 weight percent of the nanoparticle. In particular embodiments, the plasticizer encompasses about 10 weight percent of the nanoparticle. In a particular embodiment, the ratio of polymer to plasticizer (w/w) is about 5:1 to about 20:1, about 7.5:1 to about 15:1, about 8:1 to about 12:1, or about 10:1. In a particular embodiment, the ratio of polymer to cyclosporine (w/w) is about 2:1 to about 8:1, about 2.5:1 to about 6:1, about 3:1 to about 5:1, or about 4:1.

The nanoparticles of the instant invention may also comprise a surfactant (e.g., polyvinyl alcohol) to facilitate their dispersion and stability in the topical formulation (e.g., surfactant emulsifier). These surface-associated surfactants/emulsifier can be anionic (e.g., sodium dodecyl sulfate, sodium dodecyl benzene sulfonate, sodium laureth sulfate, sodium lauroyl sarcosinate, sodium myreth sulfate, sodium pareth sulfate, sodium stearate, etc.), neutral (e.g., poly vinyl alcohol, ethoxylated aliphatic alcohol, polyoxyethylene surfactants, carboxylic esters, polyethylene glycol esters, anhydrosorbitol ester and ethoxylated derivatives thereof, glycol esters of fatty acids, carboxylic amides, monoalkanolamine condensates, polyoxyethylene fatty acid amides), or cationic (e.g., quaternary ammonium salts, amines with amide linkages, polyoxyethylene alkyl and alicyclic amines, N,N,N′,N′ tetrakis substituted ethylenediamines, 2-alkyl 1-hydroxethyl 2-imidazolines); amphoteric type (e.g., amphoteric surfactants contains both an acidic and a basic hydrophilic moiety in their surface, N-coco 3-aminopropionic acid/sodium salt, N-tallow 3-iminodipropionate disodium salt, N-carboxymethyl N dimethyl N-9 octadecenyl ammonium hydroxide, N-cocoamidethyl-N-hydroxyethylglycine sodium salt.

In a particular embodiment, the nanoparticles of the instant invention comprise PLGA, dimethyl tartrate, poly vinyl alcohol, and cyclosporine.

Methods of synthesizing the nanoparticles are also encompassed by the instant invention. The nanoparticles of the instant invention may be synthesized by known methods. Methods for synthesizing nanoparticles are provided in U.S. Pat. Nos. 7,332,159; 10,517,934; Adjei et al. (2014) Nanomedicine, 9:267-278; Singhal et al. (2013) Cell Death Dis., 4:e903; and Reddy et al. (2009) FASEB J., 23(5): 1384-1395 (each of these references is incorporated by reference herein). In a particular embodiment, the nanoparticles of the instant invention are synthesized by an emulsion solvent evaporation method. In a particular embodiment, the nanoparticles of the instant invention are synthesized by a solid-in-oil-in-water emulsion method (e.g., Toorisaka, et al. (2018) J. Encapsul. Adsorp. Sci., 8:58-66; incorporated herein by reference). For example, a water and drug (e.g., hydrophilic drug (e.g., minoxidil)) in oil emulsion may be prepared and then lyophilized. The resultant solid may then be used in nanoparticle preparation. The nanoparticles may also be purified after synthesis by methods known in the art. For example, the nanoparticles may be purified by size exclusion chromatography (e.g., using a Sephacryl™ column) and/or centrifugal filtration (e.g., using a molecular weight cutoff filter). In a particular embodiment, the nanoparticles are purified such that at least 95%, 96%, 97%, 98%, 99%, or more of undesired components are removed from the sample.

In a particular embodiment, the synthesis method comprises an oil in water emulsion wherein the oil phase comprises PLGA, dimethyl tartrate, and cyclosporine and the aqueous phase comprises poly vinyl alcohol and deionized water.

The nanoparticles of the instant invention may be delivered to a subject at various concentrations. In a particular embodiment, the nanoparticles are delivered to a subject at a concentration up to about 1000 μg/ml, up to about 800 μg/ml, or up to about 600 μg/ml.

In accordance with another aspect of the instant invention, compositions comprising the nanoparticles of the instant invention are provided. In a particular embodiment, the composition is a topical composition (e.g., for application to the skin or eye). The compositions of the instant invention comprise at least one nanoparticle and at least one carrier (e.g., a carrier acceptable for topical delivery (e.g., a carrier acceptable for skin or ocular application; e.g., a pharmaceutically and/or cosmetically acceptable carrier). The composition may contain a skin permeation enhancer (e.g., surfactants (e.g., polysorbates, CTAB, DMAB), solvents (e.g., benzyl alcohol, isopropyl alcohol)), moisturizer, lubricant, color, dye, etc. The compositions (e.g., topical compositions) of the present invention may be made into a wide variety of product types such as, without limitation, liquids, drops, lotions, powders, creams, salves, gels, foams, milky lotions, sticks, sprays (e.g., pump spray), aerosols, ointments, pastes, mousses, dermal patches, adhesives (e.g., adhesive tape), bandages, pad, scaffold, nanofibers, films, cleansing agent, controlled release devices, and other equivalent forms. In a particular embodiment, the composition is a lotion or cream product. In a particular embodiment, the composition is a liquid or drop (e.g., eye drop) product. In some embodiments, the composition is a hair care or body care product such as, without limitation, a hair shampoo, hair conditioner, hair foam, hair spray, lotion, gel, cream, ointment, soap, powder, or a sprayable powder.

Acceptable carriers can be, without limitation, sterile liquids, such as water (may be deionized), alcohol (e.g., ethanol, isopropanol, benzyl alcohol), oils (including those of petroleum, animal, vegetable or synthetic origin, such as peanut oil, soybean oil, mineral oil, sesame oil and the like), and other organic compounds or copolymers. Water or aqueous saline solutions and aqueous dextrose and glycerol solutions may also be employed as carriers. Suitable carriers and other agents of the compositions of the instant invention are described in “Remington's Pharmaceutical Sciences” by E. W. Martin (Mack Pub. Co., Easton, PA) and “Remington: The Science and Practice of Pharmacy” by Alfonso R. Gennaro (Lippincott Williams & Wilkins) (each of the foregoing references being incorporated herein by reference). Additional general types of acceptable topical carriers include, without limitation, emulsions (e.g., microemulsions and nanoemulsions), gels (e.g., an aqueous, alcohol, alcohol/water, or oil (e.g., mineral oil) gel using at least one suitable gelling agent (e.g., natural gums, acrylic acid and acrylate polymers and copolymers, cellulose derivatives (e.g., hydroxymethyl cellulose and hydroxypropyl cellulose), and hydrogenated butylene/ethylene/styrene and hydrogenated ethylene/propylene/styrene copolymers), solids (e.g., a wax-based stick, soap bar composition), or powder (e.g., bases such as talc, lactose, starch, and the like), spray, and liposomes (e.g., unilamellar, multilamellar, and paucilamellar liposomes, optionally containing phospholipids). The acceptable carriers also include stabilizers, penetration enhancers, chelating agents (e.g., EDTA, EDTA derivatives (e.g., disodium EDTA and dipotassium EDTA), iniferine, lactoferrin, and citric acid), and excipients. Protocols and procedures which facilitate formulation of the topical compositions of the invention can be found, for example, in Cosmetic Bench Reference (Cosmetics & Toiletries, Allured Publishing Corporation, Illinois) and in International Cosmetic Ingredient Dictionary and Handbook (15^{th} Ed.) (each of the foregoing references being incorporated herein by reference).

The compositions of the instant invention may be aqueous or anhydrous. In a particular embodiment, the composition is anhydrous (e.g., anhydrous serum). In a particular embodiment, the composition is silicone-based (e.g., comprising polysilicone-11 and/or cyclopentasiloxane (e.g., Gransil GCM-5)). In a particular embodiment, the composition comprises from about 0.001% to about 5.0% nanoparticles, about 0.001% to about 1.0% nanoparticles, or about 0.005 to 0.5% nanoparticles (e.g., by weight).

As stated hereinabove, the compositions of the instant invention may further comprise at least one other agent (e.g., therapeutic agent) in addition to the nanoparticles. Alternatively, the other agent (e.g., therapeutic agent) may be contained within another separate composition from the nanoparticles of the instant invention. The compositions may be administered at the same time or at different times (e.g., sequentially). In a particular embodiment, to achieve sequential delivery, the product can be developed in the form of layers (e.g., in bandage or scaffold). The agents may be incorporated in oil phase or water phase or in both.

These nanoparticles may be employed therapeutically under the guidance of a physician or other healthcare professional or self-administered by the subject/patient. The pharmaceutical preparation comprising the nanoparticles of the invention may be conveniently formulated for administration with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of nanoparticles in the chosen medium may depend on the hydrophobic or hydrophilic nature of the medium, as well as the size, enzyme activity, and other properties of the nanoparticles. Solubility limits may be easily determined by one skilled in the art.

As used herein, “acceptable medium” or “carrier” includes any and all solvents, dispersion media and the like which may be appropriate for the desired route of administration of the preparation, as exemplified in the preceding discussion. In a particular embodiment, the carrier is an anhydrous carrier. In a particular embodiment, the carrier is for topical application and is a pharmaceutically acceptable carrier or a cosmetically acceptable carrier. The use of such media for active substances is known in the art. Except insofar as any conventional media or agent is incompatible with the nanoparticles to be administered, its use in the pharmaceutical preparation is contemplated.

The dose and dosage regimen of a nanoparticle according to the invention that is suitable for administration to a particular subject may be varied considering the patient's age, sex, weight, general medical condition, and the specific condition for which the nanoparticle is being administered and the severity thereof. The route of administration of the nanoparticle, the pharmaceutical carrier with which the nanoparticle is combined, and the nanoparticle's biological activity may also be considered.

Selection of a suitable pharmaceutical preparation may also depend upon the mode of administration chosen. For example, the nanoparticles of the invention may be administered topically. In these instances, the pharmaceutical preparation comprises the nanoparticles dispersed in a medium that is compatible with the site of administration (e.g., skin or eye). In a particular embodiment, the nanoparticles may also be injected into skin layers either using needle or diffused through the skin layers using ultrasound/UV rays/permeability enhancers or physical and mechanical techniques. As explained hereinabove, pharmaceutical preparations for topical administration are known in the art. The lipophilicity of the nanoparticles or the pharmaceutical preparation in which they are delivered may be increased so that the molecules can arrive at their target location. Methods for increasing the lipophilicity of a molecule are known in the art.

Pharmaceutical compositions containing a nanoparticle of the present invention as the active ingredient in intimate admixture with a pharmaceutical carrier can be prepared according to conventional pharmaceutical compounding techniques. The carrier may take a wide variety of forms depending on the form of preparation desired for administration, e.g., topically. A pharmaceutical preparation of the invention may be formulated in dosage unit form for ease of administration and uniformity of dosage. Dosage unit form, as used herein, refers to a physically discrete unit of the composition appropriate for the subject using the nanoparticles of the instant invention. Each dosage should contain a quantity of active ingredient calculated to produce the desired effect in association with the selected carrier. Procedures for determining the appropriate dosage unit are well known to those skilled in the art. Appropriate concentrations for alleviation of a particular pathological condition may be determined by dosage concentration curve calculations, as known in the art.

In accordance with the present invention, the appropriate dosage unit for the administration of nanoparticles may be determined by evaluating the toxicity of the molecules in animal models. Various concentrations of nanoparticle pharmaceutical preparations may be administered to mice or other mammals, and the minimal and maximal dosages may be determined based on the beneficial results and side effects observed as a result of the treatment. Appropriate dosage unit may also be determined by assessing the efficacy of the nanoparticles treatment in combination with other standard drugs. The dosage units of nanoparticles may be determined individually or in combination with each treatment according to the effect detected.

The pharmaceutical preparation comprising the nanoparticles may be administered at appropriate intervals, for example, at least twice a day or more until the pathological symptoms are reduced or alleviated, after which the dosage may be reduced to a maintenance level. The appropriate interval in a particular case would normally depend on the condition of the patient. The preparation may also be administered “as needed.”

### Definitions

The following definitions are provided to facilitate an understanding of the present invention:

The singular forms “a,” “an,” and “the” include plural referents unless the context clearly dictates otherwise.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition resulting in a decrease in the probability that the subject will develop the condition.

As used herein, the term “subject” refers to an animal, particularly a mammal, particularly a human.

A “therapeutically effective amount” of a compound or a pharmaceutical composition refers to an amount effective to prevent, inhibit, treat, or lessen the symptoms of a particular disorder or disease. The treatment of inflammation or infection herein may refer to curing, relieving, and/or preventing the inflammation or infection, the symptom(s) of it, or the predisposition towards it.

As used herein, the term “therapeutic agent” refers to a chemical compound or biological molecule including, without limitation, nucleic acids, peptides, proteins, and antibodies that can be used to treat a condition, disease, or disorder or reduce the symptoms of the condition, disease, or disorder.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, or nucleic acids, though they may be amino acids or dipeptides.

As used herein, the term “amphiphilic” means the ability to dissolve in both water and lipids/apolar environments. Typically, an amphiphilic compound comprises a hydrophilic portion and a hydrophobic portion. “Hydrophobic” designates a preference for apolar environments (e.g., a hydrophobic substance or moiety is more readily dissolved in or wetted by non-polar solvents, such as hydrocarbons, than by water). As used herein, the term “hydrophilic” means the ability to dissolve in water.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Polysorbate 80), emulsifier, buffer (e.g., Tris HCl, acetate, phosphate), bulking substance (e.g., lactose, mannitol), excipient, auxiliary agent or vehicle with which an active agent of the present invention is administered. Pharmaceutically or cosmetically acceptable carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. The compositions can be incorporated into particulate preparations of polymeric compounds such as polylactic acid, polyglycolic acid, etc., or into liposomes or micelles. Such compositions may influence the physical state, stability, rate of in vivo release, and rate of in vivo clearance of components of a pharmaceutical composition of the present invention. The pharmaceutical composition of the present invention can be prepared, for example, in liquid form, or can be in dried powder form (e.g., lyophilized). Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

As used herein, the term “purified” or “to purify” refers to the removal of contaminants or undesired compounds from a sample or composition. For example, purification can result in the removal of from about 70 to 90%, up to 100%, of the contaminants or undesired compounds from a sample or composition. In certain embodiments, at least 90%, 93%, 95%, 96%, 97%, 98%, 99%, 99.5%, or more of undesired compounds from a sample or composition are removed from a preparation.

Hair regrowth agents are agents that promote hair regrowth and/or hair thickness. In some embodiments, the hair regrowth agent promotes the transition of vellus hair to terminal hair; increases vellus and/or terminal hair regrowth; maintains terminal hair regrowth; and/or prevents and/or inhibits miniaturization of terminal hairs. Examples of hair regrowth are provided, for example, in Gensure, R. (Chapter 4, “Pharmacological Treatment of Alopecia” in Alopecia, Ed. M. Ahmad, IntechOpen, 2018, DOI: 10.5772/intechopen.79656), incorporated by reference herein. These examples include, without limitation, spironolactone, minoxidil, finasteride, oral contraceptives, glucocorticoids, Janus kinase (JAK) inhibitors (e.g., tofacitinib or ruxolitinib), bimatoprost, diphenylcyclopropenone (DPCP), androgen receptor antagonist, vitamin D analogs, parathyroid hormone antagonists, TGF-beta receptor antagonists, anti-fibrogenic factor, neurotrophic activator, histone deacetylase inhibitor (e.g., suberohydroxamic acid phenyl ester), and interleukin antibodies (e.g., tralokinumab or secukinumab). In a particular embodiment, the hair regrowth agent is selected from the group consisting of minoxidil, 5-alpha-reductase inhibitors (e.g., finasteride, dutasteride, alfatradiol, turosteride, bexlosteride, izonsteride, and epristeride), prostamides, and prostaglandin F2a (PGF2α) analogs (e.g., bimatoprost, travoprost, latanoprost, dinoprost, carboprost, and tafluprost). In a particular embodiment, the hair regrowth agent is selected from the group consisting of minoxidil, finasteride, and bimatoprost.

Antioxidants are substances which neutralize the activity of reactive oxygen species or inhibit the cellular damage done by the reactive species or their reactive byproducts or metabolites. The term “antioxidant” may also refer to compounds that inhibit, prevent, reduce or ameliorate oxidative reactions or compounds that inhibit reactions promoted by reactive oxygen species such as oxygen itself, oxygen free radicals, or peroxides. Examples of antioxidant enzymes include, but are not limited to: superoxide dismutase (e.g., SOD1), catalase, peroxidase, glutathione peroxidase, glutathione reductase, glutathione-S-transferase, methionine sulfoxide reductase, and hemeoxygenase. For example, the antioxidant enzyme superoxide dismutase (SOD), particularly, SOD1 (also called Cu/Zn SOD), is known to catalyze the dismutation of superoxide (O₂·⁻). Examples of other antioxidants include, without limitation: Bcl-2 (B-cell lymphoma 2), plant derived antioxidants, vitamin E, vitamin C, ascorbyl palmitate, vitamin A, methionine, carotenoids, beta carotene, retinoids, xanthophylls, lutein, zeaxanthin, flavones, isoflavones, flavanones, flavonols, catechins, ginkgolides, anthocyanidins, proanthocyanidins, carnosol, carnosic acid, organosulfur compounds, allylcysteine, alliin, allicin, lipoic acid, omega-3 fatty acids, eicosapentaeneoic acid (EPA), docosahexaeneoic acid (DHA), tryptophan, arginine, isothiocyanates, quinones, ubiquinols, butylated hydroxytoluene (BHT), butylated hydroxyanisole (BHA), super-oxide dismutase mimetic (SODm), and coenzymes-Q. In a particular embodiment, the antioxidant is an antioxidant vitamin (e.g., Vitamin A, C, and/or E). In a particular embodiment, the antioxidant is an antioxidant enzyme, particularly catalase and/or methionine sulfoxide reductase (e.g., of mammalian, particularly human, origin). The antioxidant may be isolated from natural sources or prepared recombinantly.

The following examples provide illustrative methods of practicing the instant invention and are not intended to limit the scope of the invention in any way.

### Example 1

To prepare poly-lactic-co-glycolic acid (PLGA) particles loaded with cyclosporine A, an oil in water emulsion was formed via homogenization at 11000 RPM. The oil phase comprised PLGA (27.7 mg PLGA/mL), dimethyl tartrate (2.7 mg DMT/mL), and cyclosporine (6.9 mg CsA/mL) dissolved in ethyl acetate. The aqueous phase comprised poly vinyl alcohol (30 mg PVA/mL) and deionized water, optionally with 0.07 EA/ml of water. To form the emulsion, the aqueous phase was added to a beaker and the homogenizer was started. The oil phase was then slowly added to the aqueous phase. The two phases were combined in a ratio of 2 mL aqueous to 1 mL oil for the emulsion step. Homogenization continued until the particles had a hydrodynamic diameter of ˜220 nm as measured by dynamic light scattering (DLS).

The emulsion was then transferred to a rotary evaporator to remove the ethyl acetate and harden the PLGA particles. Excess PVA and un-encapsulated cyclosporine was then removed by tangential flow filtration, resulting in a purified suspension of PLGA particles in water. This suspension was then frozen and dried via lyophilization resulting in a cake of cyclosporine loaded PLGA nanoparticles.

The typical physical properties of the produced particles include:
Hydrodynamic Diameter: ˜220 nm
Poly Dispersity Index: ˜0.1-0.2
Zeta Potential: −10-−25 mV
Cyclosporine Encapsulation Efficiency: 10%-30%
Cyclosporine Composition of Particle: 50-100 μg/mg.

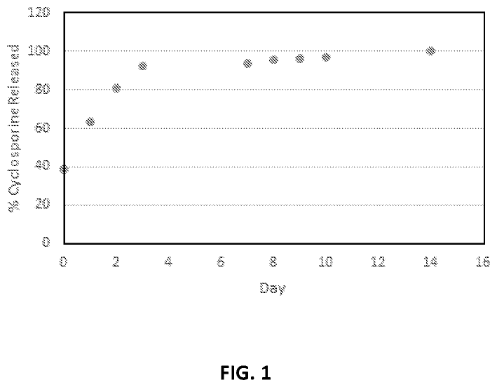

To measure the release of the cyclosporine from the particles, a sample of the dried PLGA particles was resuspended in water and incubated at 35° C. The particles were then sedimented via centrifugation at 12000 RPM for 10 minutes and the supernatant was taken for analysis. The particles were then resuspended in fresh water to continue the release. Detection and quantification of cyclosporine was performed using UPLC-UV and comparing to known standard samples. FIG. 1 provides a release profile of cyclosporine from the particles.

### Example 2

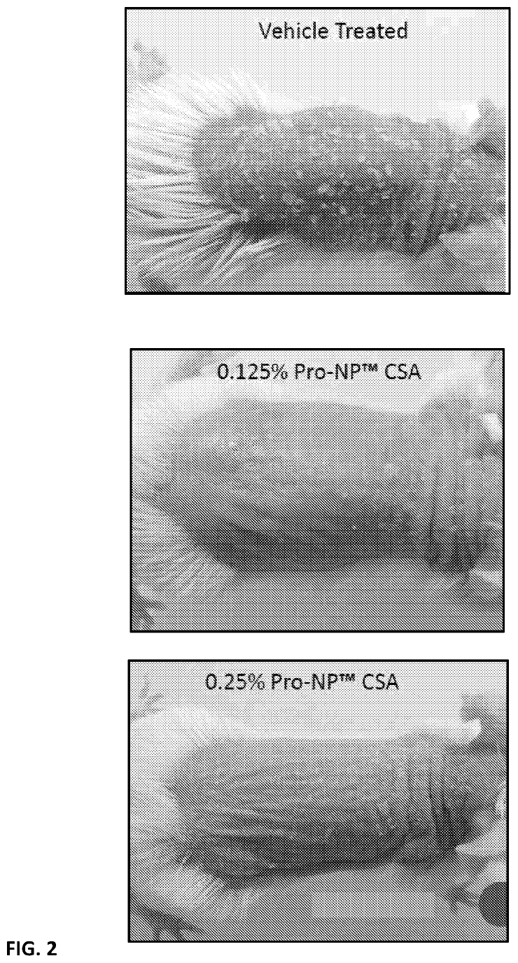

Cyclosporine A (CSA) loaded PLGA particles (Pro-NP™) were tested in a mouse model of plaque psoriasis. Topical application of imiquimod to mice induces plaque psoriasis (e.g., van der Fits, et al. (2009) J. Immunol., 182(9):5836-5845). After application of imiquimod for 7 days, 2.5% or 5% cyclosporine A particles were topically applied to the mice (n=12). Control mice were treated with vehicle/placebo. As seen in FIG. 2, topical cyclosporine A significantly improved imiquimod induced plaque psoriasis in the mouse model. Indeed, plaque severity scoring returned to baseline, nearly eliminating plaque psoriasis in 7 days. Further, secondary measures of inflammation and skin integrity returned to baseline and quality of life metrics significantly improved with topical treatment of cyclosporine particles.

Based on the results in the mouse model, a prospective randomized blinded controlled clinical pilot study was performed for evaluating the efficacy of cyclosporine A particles versus placebo (carrier) for the treatment of chronic stable plaque psoriasis. Pro-NP™ nanoparticle-encapsulated CSA (0.25%) was provided in a serum carrier for topical application once daily for 12 weeks. Four patients were enrolled in the study with three receiving the active drug and one receiving a placebo/vehicle. Standard blood panels indicated that all patients had normal kidney and liver function through the study.

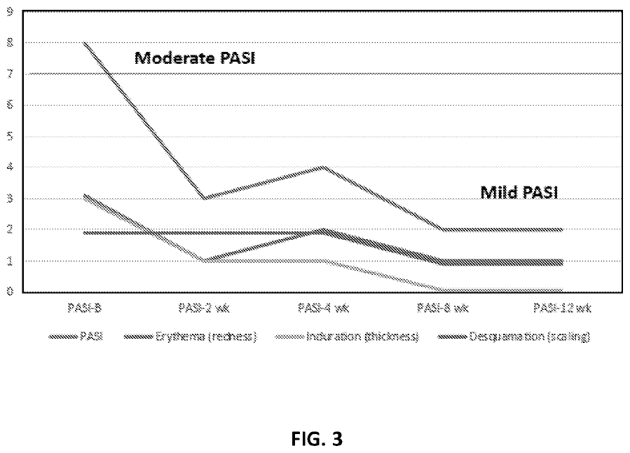

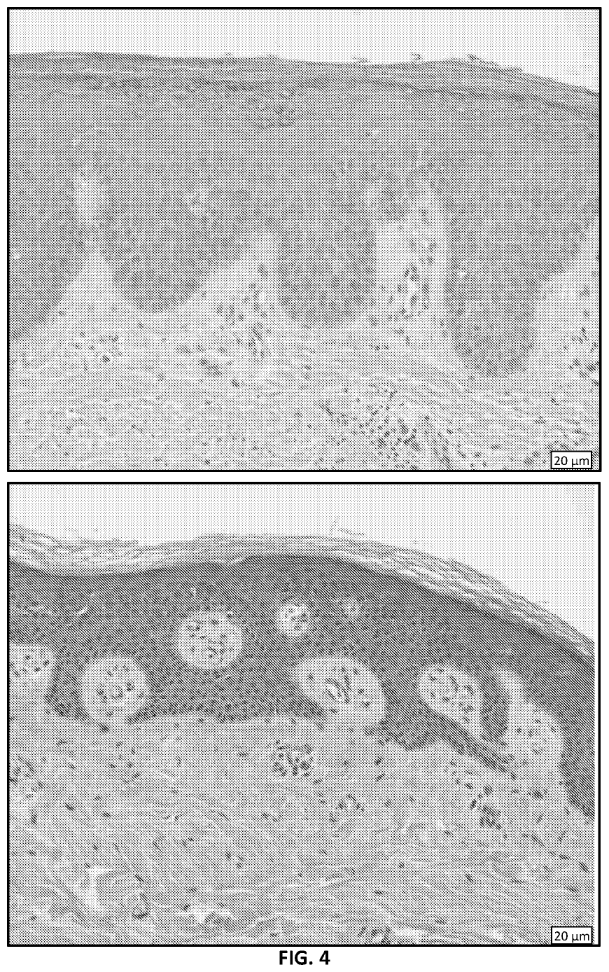

The psoriasis of the patients was assessed by Dermatology Life Quality Index (DLQI), Target Lesion Severity Score (TLSS—a composite of redness, scaling, and plaque elevation), the Psoriasis Area and Severity Index (PASI—a composite of redness, thickness, and scaling) and photography. As seen in Table 1, administration of cyclosporine A particles significantly reduced the psoriasis in the subjects. Indeed, two of the subjects saw significant improvement in PASI and TLSS with reduction from severe psoriasis to mild or moderate during the 12 week treatment. Notably, patient 3 receiving the active drug showed dramatic improvement in plaque psoriasis with improvements in erythema, induration, and desquamation (FIG. 3). As seen in FIG. 4, patient 3 had significantly improved skin biopsies after treatment with a loss of psoriasiform hyperplasia and a more normal epidermis. There was also decreased inflammatory infiltrate with CD-4 and CD-8.

TABLE 1
PASI (clear = 0, mild = 1-6, moderate = 7-9),
TLSS (clear = 0, slight to mild = 1-3, moderate =
4-6, severe = 7-9), and DLQI at final visit compared
to first are provided for the 4 treated patients.

| Active/ | PASI | PASI | TLSS | TLSS | |
|---|---|---|---|---|---|
| Placebo | Initial | Final | Initial | Final | DLQI |
| Active (1) | 8 | 6 | 9 | 5 | Improved |
| Active (2) | 6 | 7 | 6 | 6 | Improved |
| Active (3) | 8 | 2 | 8 | 2 | Improved |
| Placebo | 4 | 2 | 4 | 2 | Worsened |

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A nanoparticle comprising cyclosporine, a biodegradable polymer, a surfactant emulsifier, and a plasticizer.

2. The nanoparticle of claim 1, wherein said biodegradable polymer is poly-lactic-co-glycolic acid.

3. The nanoparticle of claim 1, wherein said plasticizer is dimethyl tartrate.

4. The nanoparticle of claim 1, wherein said emulsifier is polyvinyl alcohol.

5. The nanoparticle of claim 1 comprising cyclosporine, poly-lactic-co-glycolic acid, dimethyl tartrate, and polyvinyl alcohol.

6. A composition comprising the nanoparticle of claim 1 and a pharmaceutically acceptable carrier.

7. A method of treating, inhibiting, and/or preventing hair loss and/or regrowing and/or thickening hair in a subject in need thereof, said method comprising topically administering the nanoparticle of claim 1 to the skin of the subject.

8. The method of claim 7, wherein said hair loss is caused by alopecia, alopecia areata, androgenetic alopecia, hypotrichosis, or hair miniaturization.

9. The method of claim 7, further comprising administering at least one other hair regrowth agent or at least one antioxidant.

10. A method of treating, inhibiting, and/or preventing psoriasis in a subject in need thereof, said method comprising topically administering the nanoparticle of claim 1 to the skin of the subject.

11. A method of treating, inhibiting, and/or preventing arthritis in a subject in need thereof, said method comprising topically administering the nanoparticle of claim 1 to the skin of the subject.

12. A method of treating, inhibiting, and/or preventing dry eye disease in a subject in need thereof, said method comprising administering the nanoparticle of claim 1 to the eye of the subject.
